(19) United States
(12) Patent Application Publication
(10) Pub. No.: US 2019/0194610 A1
(43) Pub. Date: Jun. 27, 2019

(54) METHODS AND COMPOSITIONS FOR IN VIVO TRANSGENESIS

(71) Applicant: Massachusetts Institute of Technology, Cambridge, MA (US)

(72) Inventor: Kevin Esvelt, Newton, MA (US)

(21) Appl. No.: 16/178,696

(22) Filed: Nov. 2, 2018

Related U.S. Application Data
(60) Provisional application No. 62/581,234, filed on Nov. 3, 2017.

(51) Int. Cl. C12N 5/075; C07K 14/71

(52) U.S. Cl. CPC C12N 5/0609; C07K 14/71

(57) ABSTRACT

The invention relates, in part, to methods to deliver molecules into in vitro and in vivo cells and into organisms using exogenous and/or endogenous membrane proteins.

### RELATED APPLICATIONS

This application claims benefit under 35 U.S.C. § 119(e) of U.S. Provisional Application Ser. No. 62/581,234 filed Nov. 3, 2017, the disclosure of which is incorporated by reference herein in its entirety.

### GOVERNMENT INTEREST

This invention was made with government support under grant number DP2 AI136597-01 awarded by the National Institutes of Health. The Government has certain rights in the invention.

### FIELD OF THE INVENTION

The invention relates, in part, to methods of preparing cells, genomes, and organisms that include genome modifications and relates, in part, to methods and compositions for delivering molecules into cells.

### BACKGROUND OF THE INVENTION

Current approaches do not offer efficient in vivo transgenesis; they typically require the death of the animal, are not very efficient, and often result in mosaics. Germline targeting creates heterozygote transgenics in the first generation and they do not permit delivery of large amounts of DNA.

### SUMMARY OF THE INVENTION

According to one aspect of the invention, compositions that include a host cell are provided, the host cell including: a preselected biomolecule expressed on the surface of the host cell, wherein the expressed preselected exogenous biomolecule is capable of enabling delivery of one or more preselected agents into the host cell; and one or more preselected agents comprising at least one of a: nucleic acid molecule, DNA molecule, RNA molecule, nucleic acid-editing molecule, and genome-editing molecule. In some embodiments, the preselected biomolecule is an exogenous biomolecule that is not naturally expressed in the host cell. In certain embodiments, the preselected biomolecule is an endogenous biomolecule that is naturally expressed in the host cell. In some embodiments, the preselected biomolecule comprises a receptor molecule, optionally, wherein the receptor molecule comprises one or more of: a low-density lipoprotein (LDL) receptor, a very low-density lipoprotein receptor a membrane channel, and a membrane pore. In some embodiments, the LDL receptor is a vitellogenin receptor. In certain embodiments, the expressed preselected biomolecule on the surface of the host cell is capable of selectively interacting with a ligand molecule. In some embodiments, the composition further comprises a ligand complex comprising the ligand molecule and at least one preselected agent in association with the ligand molecule. In some embodiments, the ligand molecule is attached to at least one first preselected exogenous agent capable of associating with a second preselected exogenous agent, and wherein the interaction of the ligand molecule with the expressed preselected biomolecule is capable of enabling delivery of the associated second preselected exogenous agent into the host cell. In some embodiments, the first preselected agent comprises streptavidin and the second preselected agent comprises a biotinylated DNA molecule. In certain embodiments, the receptor molecule includes one or more of: a low-density lipoprotein receptor, a very low density lipoprotein receptor, a membrane channel, and a membrane pore. In some embodiments, the ligand complex comprises a ligand molecule associated with at least one first preselected agent capable of associating with a second preselected agent and wherein the interaction of the ligand molecule with the expressed preselected biomolecule is capable of enabling delivery of the associated second preselected agent into the host cell. In some embodiments, the receptor molecule comprises a vitellogenin receptor and the ligand molecule comprises a vitellogenin ligand. In certain embodiments, the vitellogenin ligand is attached to at least one preselected agent. In some embodiments, the ligand complex comprises a peptide ligand molecule attached to a preselected agent comprising a nucleic acid-editing molecule. In some embodiments, the nucleic acid-editing molecule is a DNA-editing molecule. In certain embodiments, the nucleic acid-editing molecule is one or more of a: Cas9 molecule, CRISPR nuclease, nickase, nuclease, integrase, base editor molecule, enzyme, and recombinase. In some embodiments, the first preselected agent comprises a streptavidin molecule and the second preselected agent comprises a biotinylated molecule. In some embodiments, the biotinylated molecule is a biotinylated nucleic acid molecule. In some embodiments, the biotinylated molecule is a biotinylated DNA molecule. In certain embodiments, the receptor molecule comprises a virus receptor capable of attaching a virus to the host cell. In some embodiments, the virus receptor is an exogenous virus receptor that is at least one of: (i) not naturally expressed in a cell of the species, (ii) a synthetic receptor, and (iii) expressed in a different species than the species of the host cell. In certain embodiments, the ligand molecule comprises a packaged virion capable of selectively interacting with the receptor molecule and delivering a preselected agent into the host cell. In some embodiments, the preselected agent comprises at least one nucleic acid molecule. In some embodiments, the packaged virion is an HSV packaged virion. In certain embodiments, the host cell also includes at least one nucleic acid-editing molecule. In some embodiments, the nucleic acid-editing molecule comprises at least one of a nucleic acid molecule and a DNA-editing molecule. In some embodiments, the host cell is in an organism. In certain embodiments, the organism comprises a plurality of host cells expressing the preselected biomolecule on their surfaces. In some embodiments, the one or more of the plurality of host cells comprises genome edits comprising an insertion of an exogenous nucleic acid into the genome of the host cell. In some embodiments, the host cell is a germline cell. In some embodiments, the host cell is a somatic host cell. In certain embodiments, the preselected agent comprises one or more of a: nucleic acid, DNA, RNA, polypeptide, chemical, toxin, enzyme, nucleic-acid editing molecule, genome-editing molecule, therapeutic molecule, and detectable label molecule. In some embodiments, the one or more nucleic acid-editing molecules comprise at least one of an: enzyme, recombinase, nuclease, nickase, integrase, CRISPR nuclease, and base editor molecule. In some embodiments, the preselected agent is an exogenous agent. In certain embodiments, the association comprises an attachment. In some embodiments, the host cell further comprises one or more exogenous edits in the genome of the cell.

According to another aspect of the invention, an organism is produced from a germline cell of any of the abovementioned embodiments of the invention. According to another aspect of the invention, offspring of any embodiment of any aforementioned progeny organism are provided.

According to another aspect of the invention a progeny cell of any embodiment of any aforementioned aspects of the invention is provided, wherein the progeny cell includes one or more of the genome edits of the somatic host cell.

According to another aspect of the invention, methods of delivering one or more exogenous agents into an oocyte of an original species re provided, the methods including: preparing a transgenic oocyte comprising an expressed exogenous low-density lipoprotein (LDL) receptor in a host oocyte of an original species, wherein (i) the exogenous LDL receptor is not naturally expressed in an oocyte of the original species and (ii) the expressed exogenous LDL receptor is displayed on the surface of the transgenic oocyte; contacting the expressed exogenous LDL receptor with a ligand complex comprising: (iii) an LDL receptor ligand molecule that specifically interacts with the expressed exogenous LDL receptor and (iv) one or more preselected agents in association with the LDL receptor ligand molecule, wherein the contacted expressed exogenous LDL receptor is capable of enabling delivery of the one or more preselected agents into the transgenic oocyte; and delivering the one or more preselected agents into the transgenic oocyte displaying the expressed exogenous LDL receptor. In certain embodiments, the LDL receptor ligand molecule is in association with at least one first preselected agent that is in association with a second preselected agent, wherein the specific interaction of the LDL receptor ligand molecule with the expressed exogenous LDL receptor delivers the second preselected agent into the transgenic oocyte. In some embodiments, the first preselected agent comprises a streptavidin molecule and the second preselected agent comprises a biotinylated molecule. In some embodiments, the expressed exogenous LDL receptor comprises an exogenous vitellogenin receptor and the LDL receptor ligand molecule comprises a vitellogenin receptor ligand that specifically interacts with the expressed exogenous vitellogenin receptor. In certain embodiments, the vitellogenin receptor ligand is attached to at least one preselected agent and the interaction between the expressed exogenous vitellogenin receptor molecule and the vitellogenin receptor ligand delivers at least one of the preselected agents into the transgenic oocyte. In some embodiments, the vitellogenin receptor ligand is attached to at least one first preselected agent that is in association with a second preselected agent and the interaction of the vitellogenin receptor ligand with the expressed preselected exogenous vitellogenin receptor delivers the second preselected agent into the transgenic oocyte. In some embodiments, at least one of the preselected first agents comprises a streptavidin molecule and at least one of the preselected second agents comprises a biotinylated molecule. In some embodiments, the biotinylated molecule comprises a nucleic acid molecule, optionally a DNA molecule. In certain embodiments, the preselected agent comprises at least one of a: nucleic acid, DNA, RNA, polypeptide, chemical, toxin, enzyme, nucleic-acid editing molecule, genome-editing molecule, therapeutic molecule, and detectable label molecule. In some embodiments, the nucleic acid-editing molecule is a DNA-editing molecule, and optionally is one of or more of a: Cas9 molecule, CRISPR nuclease, nickase, nuclease, integrase, base editor molecule, enzyme, and recombinase. In some embodiments, the method also includes delivering at least one nucleic acid and nucleic acid-editing molecule into the transgenic oocyte, optionally wherein the nucleic acid-editing molecule is a genome-editing molecule. In certain embodiments, a means of delivering the nucleic acid-editing molecule comprises delivering into the transgenic oocyte a nucleic acid molecule encoding the nucleic acid-editing molecule, and expressing the nucleic acid-editing molecule in the transgenic oocyte. In some embodiments, the nucleic acid encoding the nucleic acid-editing molecule further comprises a nucleic acid encoding a molecule to be inserted into the genome of the transgenic oocyte. In some embodiments, at least one nucleic acid molecule and one nucleic acid-editing molecule are delivered into the transgenic oocyte as a single fused molecule. In some embodiments, the fused molecule comprises (i) a molecule to be inserted into the genome of the transgenic oocyte and (ii) and encodes the nucleic acid-editing molecule, wherein the encoded nucleic acid-editing molecule is expressed in the transgenic oocyte after the delivery and the molecule is inserted into the genome of the transgenic oocyte. In certain embodiments, the method also includes editing the genome of the transgenic oocyte. In some embodiments, the method also includes developing an organism from the transgenic oocyte. In some embodiments, the method also includes producing one or more offspring from the developed organism. In certain embodiments, the association comprises an attachment. In some embodiments, the nucleic acid-editing molecule is attached (fused) to a detectable label. In certain embodiments, the detectable label is a fluorescent label. In some embodiments, the organism expresses a genome editing system and a vitellogenin receptor that enables delivery of one or more of: guide RNAs, DNA for insertion into the genome of the progeny, and DNA that encodes one or more guide RNAs. In certain embodiments, the genome editing system comprises a CRISPR enzyme. In some embodiments, the genome editing system comprises a Cas9 system.

According to another aspect of the invention, methods of delivering one or more exogenous agents into an oocyte of an original species are provided, the methods including: contacting a host oocyte displaying an expressed endogenous low-density lipoprotein (LDL) receptor on its surface, with a ligand complex comprising: (i) an LDL receptor ligand molecule that specifically interacts with the expressed endogenous LDL receptor and (ii) one or more preselected agents in association with the LDL receptor ligand molecule, wherein the contacted expressed endogenous LDL receptor is capable of enabling delivery of the one or more preselected agents into the host oocyte; and delivering the one or more preselected agents into the host oocyte displaying the expressed exogenous LDL receptor on its surface. In some embodiments, the LDL receptor ligand molecule is in association with at least one first preselected agent that is in association with a second preselected agent, wherein the specific interaction of the LDL receptor ligand molecule with the expressed endogenous LDL receptor delivers the second preselected agent into the host oocyte. In some embodiments, the first preselected agent comprises a streptavidin molecule and the second preselected agent comprises a biotinylated molecule. In some embodiments, the expressed endogenous LDL receptor comprises an endogenous vitellogenin receptor and the LDL receptor ligand molecule comprises an endogenous vitellogenin receptor ligand that specifically interacts with the expressed endogenous vitellogenin receptor. In certain embodiments, the vitellogenin receptor ligand is attached to at least one preselected agent and the interaction between the expressed endogenous vitellogenin receptor molecule and the vitellogenin receptor ligand delivers at least one of the preselected agents into the host oocyte. In some embodiments, the vitellogenin receptor ligand is attached to at least one first preselected agent that is in association with a second preselected agent and the interaction of the vitellogenin receptor ligand with the expressed endogenous vitellogenin receptor delivers the second preselected agent into the host oocyte. In some embodiments, at least one of the preselected first agents comprises a streptavidin molecule and at least one of the preselected second agents comprises a biotinylated molecule. In some embodiments, the biotinylated molecule comprises a nucleic acid molecule, optionally a DNA molecule. In certain embodiments, the preselected agent comprises at least one of a: nucleic acid, DNA, RNA, polypeptide, chemical, toxin, enzyme, nucleic-acid editing molecule, genome-editing molecule, therapeutic molecule, and detectable label molecule. In some embodiments, the nucleic acid-editing molecule is a DNA-editing molecule, and optionally is one of or more of a: Cas9 molecule, CRISPR nuclease, nickase, nuclease, integrase, base editor molecule, enzyme, and recombinase. In some embodiments, the method also includes delivering at least one nucleic acid and at least one nucleic acid-editing molecule into the host oocyte, optionally wherein the nucleic acid-editing molecule is a genome-editing molecule. In some embodiments, a means of delivering the nucleic acid-editing molecule comprises delivering into the host oocyte a nucleic acid molecule encoding the nucleic acid-editing molecule, and expressing the nucleic acid-editing molecule in the host oocyte. In certain embodiments, the nucleic acid encoding the nucleic acid-editing molecule further comprises a nucleic acid encoding a molecule to be inserted into the genome of the host oocyte. In some embodiments, at least one nucleic acid molecule and one nucleic acid-editing molecule are delivered into the host oocyte as a single fused molecule. In some embodiments, the fused molecule comprises (i) a molecule to be inserted into the genome of the transgenic oocyte and (ii) and encodes the nucleic acid-editing molecule, wherein the encoded nucleic acid-editing molecule is expressed in the host oocyte after the delivery and the molecule is inserted into the genome of the host oocyte. In certain embodiments, the method also includes editing the genome of the contacted host oocyte. In some embodiments, the method also includes developing an organism from the contacted host oocyte. In some embodiments, the methods also include producing one or more offspring from the developed organism. In some embodiments, the association comprises an attachment. In certain embodiments, the nucleic acid-editing molecule is attached (fused) to a detectable label. In some embodiments, the detectable label is a fluorescent label.

According another aspect of the invention, methods of preparing a transgenic cell are provided, the methods including: expressing a preselected biomolecule on the surface of a host cell of a species, wherein the preselected biomolecule is an exogenous molecule that is not naturally expressed on the surface of a cell of the species, and wherein the expressed preselected biomolecule is capable of enabling delivery of one or more preselected agents into the host cell. In some embodiments, the methods also include delivering one or more independently preselected agents into the host cell displaying the expressed preselected biomolecule. In some embodiments, the preselected biomolecule comprises a receptor molecule. In certain embodiments, the receptor molecule is not an endogenous receptor molecule. In some embodiments, the expressed preselected biomolecule on the surface of the host cell is capable of selectively interacting with a ligand molecule. In some embodiments, a ligand complex comprises the ligand molecule and the interaction of the ligand molecule and the expressed preselected biomolecule delivers at least a portion of the ligand complex into the host cell. In certain embodiments, the ligand complex further comprises at least one preselected agent associated with the ligand molecule. In some embodiments, the preselected agent comprises one or more of a: nucleic acid, DNA, RNA, polypeptide, chemical, toxin, enzyme, nucleic-acid editing molecule, genome-editing molecule, therapeutic molecule, and detectable label molecule. In some embodiments, the ligand molecule is attached to at least one first preselected agent capable of associating with a second preselected agent, and wherein the interaction of the ligand molecule with the expressed preselected biomolecule delivers the associated second preselected agent into the host cell. In certain embodiments, the first preselected agent comprises streptavidin and the second preselected agent comprises a biotinylated molecule. In some embodiments, the receptor molecule comprises one or more of: a low-density lipoprotein receptor, a very low-density lipoprotein receptor a membrane channel, and a membrane pore. In some embodiments, the receptor molecule includes a vitellogenin receptor and the ligand molecule comprises a vitellogenin ligand. In certain embodiments, the vitellogenin ligand is attached to at least one preselected agent and the interaction between the receptor molecule and the vitellogenin ligand delivers the at least one preselected agent into the host cell. In some embodiments, the ligand complex comprises a peptide ligand molecule attached to a preselected agent comprising a nucleic acid-editing molecule and the interaction between the receptor molecule and the peptide ligand molecule delivers the nucleic acid-editing molecule into the host cell. In some embodiments, the nucleic acid-editing molecule is a DNA-editing molecule. In certain embodiments, the nucleic acid-editing molecule is one or more of a: Cas9 molecule, CRISPR nuclease, nickase, nuclease, integrase, base editor molecule, enzyme, and recombinase. In some embodiments, the first preselected agent comprises a streptavidin molecule and the second preselected agent comprises a biotinylated molecule. In some embodiments, the biotinylated molecule is a biotinylated nucleic acid molecule. In certain embodiments, the biotinylated molecule is a biotinylated DNA molecule. In some embodiments, the receptor molecule comprises a virus receptor capable of attaching a virus to the host cell. In some embodiments, the virus receptor is an exogenous virus receptor that is at least one of: (i) not naturally expressed in a cell of the species, (ii) a synthetic receptor, and (iii) expressed in a different species than the species of the host cell. In some embodiments, the ligand molecule comprises a packaged virion capable of selectively interacting with the receptor molecule and delivering a preselected agent into the host cell. In certain embodiments, the preselected agent comprises at least one nucleic acid molecule and the selective interaction of the packaged virion with the virus receptor delivers the nucleic acid molecule into the host cell. In some embodiments, the packaged virion is an HSV packaged virion. In some embodiments, the method also includes delivering at least one nucleic acid-editing molecule into the host cell expressing the preselected biomolecule. In certain embodiments, the nucleic acid-editing molecule comprises at least one of a nucleic acid molecule and a DNA-editing molecule. In some embodiments, a means of delivering the nucleic acid-editing molecule into the host cell comprises delivering a nucleic acid molecule encoding the nucleic-editing molecule into the host cell, and wherein the nucleic acid-editing molecule is expressed in in the host cell. In certain embodiments, the nucleic acid-editing molecule expressed in the host cell causes part of the introduced nucleic acid molecule to be copied into the genome. In some embodiments, the host cell is in an organism. In some embodiments, the organism comprises a plurality of host cells expressing the preselected biomolecule on their surfaces. In certain embodiments, delivering the at least one nucleic acid-editing molecule into the host cell comprises administering the at least one nucleic acid-editing molecule to the organism. In some embodiments, the administration comprises injecting the at least one nucleic-editing molecule into the organism, and wherein the injected nucleic acid-editing molecule is delivered into at least one of the host cells expressing the receptor molecule. In some embodiments, the one or more nucleic acid-editing molecules comprise at least one of an: enzyme, recombinase, nuclease, nickase, integrase, CRISPR nuclease, and base editor molecule. In certain embodiments, the method also includes editing the genome of the host cell. In some embodiments, the host cell expressing the preselected biomolecule is a germline cell. In some embodiments, a progeny organism of the host germline cell comprises the genome edits of the host germline cell. In certain embodiments, the method also includes producing one or more progeny of the germline cell comprising the expressed preselected biomolecule. In some embodiments, the host cell expressing the preselected biomolecule is a somatic host cell. In some embodiments, a progeny cell of the somatic host cell comprises the genome edits of the somatic host cell. In certain embodiments, the method also includes producing one or more offspring of the organism comprising the somatic host cell. In some embodiments, the association comprises an attachment. In some embodiments, the nucleic acid-editing molecule is attached (fused) to a detectable label. In certain embodiments, the detectable label is a fluorescent label.

According to another aspect of the invention, a host cell that includes an exogenous genome editing system and express a vitellogenin receptor on its surface are provided. In some embodiments, the exogenous genome editing system comprises a CRISPR genome editing system. In some embodiments, the exogenous genome editing system comprises a Cas9 system.

According to another aspect of the invention methods of modifying the genome of any embodiment of any of the aforementioned host cells are provided, the methods including: 1) contacting the host cell comprising an exogenous genome editing system and having a vitellogenin receptor expressed on it surface with a vitellogenin receptor ligand in association with one or more preselected agents that include one or more: guide RNA molecules, DNA molecules encoding one or more guide RNA molecules, and molecules for insertion into the genome of the host cell, wherein the contacting delivers the one or more preselected agents into the host cell; and 2) modifying the genome of the host cell, wherein an activity of the delivered preselected agents and the genome editing system modifies the genome of the host cell. In some embodiments, the method also includes preparing the host cell that comprises the genome editing system. In some embodiments, the host cell is a germline cell. In some embodiments, the method also includes developing the host cell into an organism. In some embodiments, the method also includes generating progeny of the developed organism.

### BRIEF DESCRIPTION OF THE DRAWINGS

FIG. 1 is a schematic diagram illustrating an embodiment of the invention that includes in vivo male transgenesis by viral vector. The embodiment shown in FIG. 1 shows steps of identifying a suitable viral vector that is dependent on a particular receptor; expressing the receptor on cells of target species in vitro and verifying delivery. An optional step of swapping coat proteins on the vector with those of a second vector (Vector 2) and verifying delivery. The diagram also illustrates generating a transgenic organism that expresses the receipt in male germline cells—which includes injecting the vector into immature males, and then testing the sperm to assess success of the delivery. Some aspects of the invention include crossing the prepared organism with a female to generated transgenic offspring.

FIG. 2 is a schematic diagram illustrating an embodiment of the invention that includes in vivo female transgenesis by viral vector. The embodiment shown in FIG. 2 shows steps of identifying a suitable viral vector that is dependent on a particular receptor; expressing the receptor on cells of target species in vitro and verifying delivery. An optional step of swapping coat proteins on the vector with those of a second vector (Vector 2) and verifying delivery. The diagram also illustrates generating a transgenic organism that expresses the receipt in female germline cells—which includes injecting the vector into immature females, and then testing the delivery to the oocytes to assess success of the delivery. Some aspects of the invention include crossing the prepared organism with a male to generated transgenic offspring.

FIG. 3 is a schematic diagram illustrating an embodiment of the invention that includes in vivo female transgenesis in which the DNA and targeted DNA insertion molecule (for example an insertion enzyme together via female injection and oocyte uptake pathway. FIG. 3 provides non-limiting examples of a ligand that selectively interacts with the expressed preselected biomolecule (which in FIG. 3 is indicated to be a receptor) with various examples of preselected agents for delivery into the cell. Non-limiting examples shown in FIG. 3 include sgRNA, crRNA/tracrRNA, DNA insertion enzyme, Cas9, biotinylated DNA, ssDNA, dsDNA, CRISPR nuclease, streptavidin, etc. which are further described elsewhere herein.

FIG. 4 is a schematic diagram illustrating an embodiment of the invention that includes in vivo female transgenesis in which the female expresses a DNA insertion molecules (for example an insertion enzyme such as, but not limited to Cas9) in the organism's germline. In one illustrated embodiment of the invention guide RNA and DNA are delivered together via female injection and oocyte uptake pathway. The central section of FIG. 4 shows various ligand complexes that can be prepared and delivered using methods of the invention. In another illustrated embodiment of the invention shown in FIG. 4, guide RNA and DNA are delivered separately via female injection and oocyte uptake pathway. Non-limiting examples show a ligand complex that includes biotinylated guide RNA and a ligand complex that includes biotinylated DNA. The lowest section of FIG. 4 illustrates an embodiment of the invention that includes delivery of DNA sufficient for genome insertion via female injection and oocyte uptake pathway.

FIG. 5 is a schematic diagram illustrating an embodiment of the invention that includes in vivo biomolecule delivery into an organism that expresses endocytosis uptake pathway in target cells. A non-limiting example of the endocytosis uptake pathway shown in FIG. 5 is vitellogenin. In this in vivo delivery embodiment of the invention, molecules are delivered into the organism via bloodstream injection or injection in to the target tissue region, and the delivered molecules are taken up via the uptake molecule, receptor, etc. that is on the surface of the “host” cells. FIG. 5 shows non-limiting examples of molecules that can be delivered (referred to herein as preselected agents) including biotinylated molecules, biotinylated DNA, CRISPR molecules, CRISPR nuclease, etc. In addition to delivery of preselected agents into the organism and cells therein, embodiments of methods of the invention are shown that include producing transgenic offspring by delivering genome editing components to germline cells.

### DETAILED DESCRIPTION

The invention relates to methods and compositions for preparing engineered cells and organisms. One aspect of the invention includes engineering an organism to express a predetermined biomolecule on the surface of its germline cells that renders the cells susceptible to a viral vector that cannot transduce any other cells of the organism. The methods include, in some aspects, injection into the gonads results in transgenesis using the viral vector (which, if a herpesvirus, it can carry considerably more DNA than any virus that can otherwise transduce germline cells.

Another aspect of the invention includes methods that utilize the oocyte endocytosis pathway via yolk protein internalization (vitellogenin). This method may be used in all organisms that lay eggs. In addition to the vitellogenin embodiments in organisms that naturally express vitellogenin receptors, certain aspects of the invention include expression of receptors, including but not limited to vitellogenin receptors in cells in which they are not naturally expressed. Thus, certain aspects of the invention include expression of exogenous biomolecules in a host cell, wherein the exogenous biomolecules are displayed on the surface of the host cell and can be used to deliver preselected agents into the host cell. Non-limiting examples of preselected agents include nucleic acids, DNA, RNA, detectable labels, encoded polypeptides, etc. These molecules, which may be referred to herein as exogenous preselected molecules, can be used in methods of the invention to alter the genome of the host cell.

In some aspects of the invention a host cell is a germline cell. In certain aspects of the invention, a host cell is a somatic cell. In some embodiments of the invention, a host cell is a cultured cell. A host cell of the invention may be a cultured cell, may be maintained in culture, and/or may be used to generate a cell line that may be grown and/or expanded in culture. In certain aspects of the invention a host cell is prepared, at least in part, outside of an organism and then delivered into the organism. In some embodiments of the invention, a host cell is in an organism. Host cells prepared using methods of the invention can be utilized to prepare transgenic organisms and progeny of such organisms. Methods and compositions of the invention permit generation of host cells using efficient delivery methods of the invention by which preselected molecules are delivered into and utilized in host cells. Host cells prepared as described herein can be used to produce organisms that include generated cells of the invention. In some aspects, methods of the invention are applied to germline cells, enabling methods of highly efficient transgenesis in organisms, including but not limited to, organisms that do not have any known uptake pathway by which preselected agents can be delivered.

Aspects of the invention are based, in part, on the delivery into, and use of molecules in host cells and organisms that can be utilized to alter the host cells and/or organisms and progeny thereof. Cells prepared using methods of the invention can include, but are not limited to: genome-modifying molecules, therapeutic agents, detectable labels, etc. Methods and compositions of the invention may be used to edit the genome of a host (target) cell or organism into which exogenous molecules are delivered. As used herein, the terms “used” and “implemented” when used in reference to delivered molecules, means molecules re delivered it a cell and then the activity of the delivered molecules occurs in the host cell. In some aspects of the invention, molecules delivered into a host cell can be utilized to alter the genome of that host cell, which can result in an altered genome of an organism that develops from that host cell (e.g., from a host cell that is an oocyte). In addition, offspring of the organism that develops from the host cell may also include an altered genome. Methods and compositions of the invention permit use of an expressed preselected biomolecule that is present on the surface of a host cell, for delivering one or more preselected agents into the host cell.

As used herein, the term “biomolecule” when used in reference to a host cell comprises a molecule that when expressed in the host cell is displayed on the surface of the cell. In some embodiments of the invention, a biomolecule comprises a channel, a pore, and/or a receptor molecule. The term “preselected” when used in reference to a biomolecule means that the biomolecule was determined to either be naturally expressed on the surface of a host cell, or is chosen and then expressed on the surface of a host cell. A non-limiting example of a naturally occurring biomolecule is a vitellogenin receptor. In some aspects of the invention a vitellogenin receptor might be naturally expressed on a cell type that is used as a host cell and in other aspects of the invention a vitellogenin receptor may not be naturally expressed in a cell that is used as a host cell, but is an exogenous biomolecule.

As used herein, the term “exogenous” means not naturally present in a cell. For example, an exogenous vitellogenin receptor can be a vitellogenin receptor that is expressed on the surface of a host cell that doesn't naturally express that vitellogenin receptor. Other examples of exogenous biomolecules include, but are not limited to: a channel polypeptide, a pore polypeptide, a viral receptor, and other known molecules that can be expressed in a host cell membrane and function in a manner that enables entry of molecules from outside the host cell membrane into the host cell.

In some aspects of the invention, a desired molecule, referred to herein as a “preselected agent” can be delivered into a host cell by means of the preselected biomolecule that is expressed on the surface of the host cell. A preselected biomolecule used in aspects of the invention is capable of enabling delivery of one or more preselected agents into the host cell in which it is expressed. As used herein, the phrase “capable of enabling delivery” means that due to the presence of the preselected biomolecule, one or more preselected agent can be delivered into the cell. For example, though not intended to be limiting, a preselected biomolecule that comprises a viral receptor, is contacted and interacts with a packaged virion specific to that viral receptor and that interaction enables delivery of one or more nucleic acids associated with the packaged virion into the host cell. Non-limiting examples of a packaged virion is HSV, HIV, enveloped viruses, non-enveloped viruses, DNA virus, RNA virus, retroviruss, etc. A skilled artisan can use routine procedures to selected and use a packaged virion in embodiments of methods and compositions of the invention.

In another non-limiting example, the presence of a vitellogenin receptor on the surface of a host cell enables delivery of one or more preselected agents that are associated with a vitellogenin ligand that specifically interacts with the vitellogenin receptor, thereby enabling delivery of one or more of the associated preselected agents into the host cell. Non-limiting examples of biomolecules that may be used are: a low-density lipoprotein (LDL) receptor, a very low density lipoprotein receptor, a membrane channel, and a membrane pore. A non-limiting example of an LDL receptor is a vitellogenin receptor. Other preselected biomolecules, which in some aspects are receptor molecules can also be used in methods and compositions of the invention and can be selected and expressed in a host cell using routine procedures.

Aspects of the invention include delivery of one or more preselected agents into a host cell because of interaction of a preselected biomolecule expressed on the surface of a host cell with a ligand that selectively interact that that biomolecule. Aspects of the invention include the ligand molecule in association with one or more preselected agents that are of interest to be delivered into the host cell. Non-limiting examples of preselected agents that can be delivered into a host cell using methods and compounds of the invention are: a nucleic acid, a DNA, an RNA, a polypeptide, a chemical, a toxin, an enzyme, a nucleic-acid editing molecule, a genome-editing molecule, a therapeutic molecule, and a detectable label molecule. A preselected agent that is delivered into a host cell may be a polypeptide, including but not limited to an enzyme, an antibody or functional fragment of an antibody, a targeting protein, a substrate polypeptide, etc. Additional non-limiting examples of preselected agents that can be delivered using methods of the invention include: chemical agents, binding molecules, neutralizing agents, diagnostic agents, stabilizing agents, fixative agents, modulating peptides, activity enhancing polypeptides, inhibitory polypeptides, labeling molecules, fluorescent molecules, recombinant proteins, DNA, RNA, siRNA, small molecules, lipids, carbohydrates, synthetic peptides, peptoids, protein fragments, modified polypeptides, etc. Methods and compositions of the invention can be used to deliver a molecule of interest and a skilled artisan can use routine methods to identify a preselected agent and then deliver it in to a host cell using methods of the invention. Across a broad spectrum of compounds that can be delivered as agents using methods of the invention, routine methods can be used to select a preselected agent and to associate the preselected agent with a ligand that selectively interacts with the expressed biomolecule on the host cell. A non-limiting example of a type of nucleic acid-editing molecule that can be delivered into a host cell using a method of the invention is a DNA-editing molecule, which may include, but is not limited to: a Cas9 molecule, CRISPR nuclease, nickase, nuclease, integrase, base editor molecule, enzyme, and a recombinase molecule.

The terms: “selectively interacts” and “association with” are understood in the art. For example, a preselected receptor (biomolecule) will have a known ligand molecule that selectively interacts with the receptor. It is understood that a selective ligand is a molecule or compound that forms a complex with a biomolecule to serve a biological purpose. As used herein, interaction of the ligand and preselected biomolecule may form a complex that results in delivery into the host cell of one or more preselected agents that are in association with the ligand. Thus, interaction of the ligand and the biomolecule enables delivery of the one or more preselected agents into the host cell.

A preselected agent that is used in embodiments of methods and compounds of the invention may be an exogenous agent or can be an endogenous agent. As used herein, an exogenous preselected agent comprises a molecule that is not naturally present in the host cell into which it is delivered. For example, a CRISPR molecule, a DNA-editing molecule, a therapeutic compound, etc., which are not naturally present in an original cell of the host cell type but can be delivered using a method of the invention, are considered to be exogenous preselected agents. In some aspects of the invention, a preselected agent may be an endogenous molecule or compound that may be naturally present in an original cell of the host cell type.

A preselected agent used in methods and compositions of the invention is considered to be “in association with” a ligand and/or ligand complex. As used herein with respect to preselected agents and ligands, the terms “associated with”, “in association with”, and the like may mean a covalent interaction/attachment, a non-covalent interaction/attachment between the ligand and the preselected agent(s), or other known interaction/attachment that permits use of the associated agents and ligands in methods of the invention. A skilled artisan will understand how to use routine methods to associate a ligand and a preselected agent in a manner sufficient for use in methods and compositions of the invention.

In some aspects of the invention, there is a single preselected agent that is associated with a ligand and delivered into a host cell and in certain aspects of the invention 2, 3, 4, 5, 6, 7, or more preselected agents may be associated with a ligand and delivered into a host cell. In some embodiments one or more exogenous preselected agents may be in association with a single ligand and delivered into a host cell. In some embodiments one or more endogenous preselected agents may be in association with a single ligand and delivered into a host cell. In some embodiments one or more exogenous preselected agents and one or more endogenous preselected agents may be in association with a single ligand and delivered into a host cell.

In some aspects of the invention, the term “association with” is a direct association and in certain embodiments an association is an indirect association. For example, a direct association may be a direct attachment between a ligand and a preselected agent. Alternatively, an indirect association may be a ligand attached to a first preselected agent, wherein the first preselected agent is in association with a second preselected agent. Thus, when the first preselected agent is in associate with the ligand and the second preselected agent, the ligand and the second preselected agent are indirectly associated with each other. In some embodiments of the invention, interaction of receptor (biomolecule) displayed on the surface of a host cell with a ligand indirectly associated with a preselected agent results in delivery of the second preselected agent into the host cell. A non-limiting example of use of delivery of a second preselected agent into a host cell is association of streptavidin with a ligand, wherein a second preselected agent comprises a biotinylated molecule. The biotin associates with the first preselected agent and the biotinylated molecule is indirectly associated with the ligand. Interaction of the ligand with the biomolecule delivers the biotinylated molecule into the host cell. In some embodiments, the biotinylated molecule comprises one or more of: a nucleic acid molecule, a DNA molecule, an RNA molecule, or other suitable molecule to be delivered into the host cell.

### Preselected Agents

Numerous preselected agents can be delivered into a host cell using embodiments of methods and compounds of the invention. In addition to additional preselected agents described elsewhere herein, a preselected agent may be a component of a gene drive system. For example, components of gene drive systems such as, but not limited to: drive elements, guide RNAs, expression cassettes, vectors, endonucleases, promoters, DNA binding proteins, etc. can be preselected agents and delivered into host cells and utilized according to methods of the invention. Methods for selecting, preparing and using such preselected agents, are known in the art and may be used in conjunction with methods of the invention to prepare cells and organisms. Preselected agents that may be delivered and used in methods of the invention include, but are not limited to certain RNA-guided DNA-binding proteins, CRISPR system components, etc. Details of CRISPR systems such as CRISPR-Cas systems and examples of their use are known in the art, see for example: Deltcheva, E. et al. Nature 471, 602-607 (2011); Gasiunas, G., et al., PNAS USA 109, E2579-2586 (2012); Jinek, M. et al. Science 337, 816-821 (2012); Sapranauskas, R. et al. Nucleic acids research 39, 9275-9282 (2011); Bhaya, D., et al., Annual review of genetics 45, 273-297 (2011); and H. Deveau et al., Journal of Bacteriology 190, 1390 (February, 2008), the content of each of which is incorporated by reference herein in its entirety.

Three classes of CRISPR systems are generally known and are referred to as Type I, Type II or Type III and elements of such systems can be delivered into a host cell using methods of the invention. Type I, II, and III CRISPR systems and their components are well known in the art. See for example, K. S. Makarova et al., Nature Reviews Microbiology 9, 467 (June, 2011); P. Horvath & R. Barrangou, Science 327, 167 (Jan. 8, 2010); H. Deveau et al., Journal of Bacteriology 190, 1390 (February, 2008); J. R. van der Ploeg, Microbiology 155, 1966 (June, 2009), the contents of each of which is incorporated by reference herein in its entirety. Bioinformatic analyses have generated extensive databases of CRISPR loci in a variety of bacteria that may be used in conjunction with methods of the invention to select suitable preselected agents for delivery. See for example: M. Rho, et al., PLoS genetics 8, e1002441 (2012) and D. T. Pride et al., Genome Research 21, 126 (January, 2011) each of which is incorporated by reference herein in its entirety. A recently designated Type V system is similar in many aspects to Type II systems and may be relevant for genome editing and thus components of Type V systems may be delivered into a host cell of the invention (see additional information: B. Zetsche et al., 2015, Cell 163, 1-13; T. Yamano et al., 2016, Cell, April 21 doi:10.1016/j.cell.2016.04.003; D. Dong et al., 2016, Nature, 20 April, doi:10.1038/nature17944; I. Fonfara et al., 2016, Nature, 20 April, doi:10.1038/nature17945).

It will be understood that references herein to “Cas9”, the RNA-guided DNA-binding protein nuclease of Type II CRISPR systems, can be replaced by “Cpf1”, the RNA-guided DNA-binding protein nuclease of Type V systems. It will be understood, as described elsewhere herein, certain embodiments of host cells of the invention may include a targeted DNA-binding nuclease other than an RNA-guided DNA-binding nuclease. For example, in some embodiments a nucleic acid-guided DNA binding nuclease such as a DNA-guided DNA-binding nuclease may be delivered into a host cell (see Gao, F., et al., Nature Biotech online publication, May 2, 2016: doi: 10.1038/nbt.3547, the content of which is incorporated herein by reference). Certain aspects of the invention include methods of preparing cells, cell lines, and/or organisms that include host cells in which nucleic acids that encode Cas9 or other CRISPR nuclease proteins such as Cpf1.

### Gene Editing Components

Aspects of the invention include methods of preparing cells, cell lines, and/or organisms in which methods of the invention permit genome editing. In some aspects, the invention includes methods to deliver components of genome editing systems comprising components that can be separately encoded as nucleic acid sequences that are delivered into the genome a host cell or organism. Preselected components may include, but are not limited to: guide RNAs, guided DNA binding proteins, nucleic acid-guided DNA binding proteins, RNA-guided DNA binding proteins, DNA-guided DNA binding proteins, promoter/enhancer/3′UTR sequences, housekeeping gene sequences, promoter sequences, predetermined target genes, tRNA sequences, and sequences encoding detectable labels, such as but not limited to fluorescent labels.

As used herein the term “host” or “target” when used in reference to a cell, cell line or organism, means a cell, cell line, or organism, respectively that includes preselected biomolecule expressed on its surface that is capable of enabling delivery of one or more preselected agents in to the host cell. In some embodiments of the invention, a host cell is a germline cell. In certain embodiments, the host cell is a somatic cell. In some aspects of the invention, the host cell is in vitro, and may, in some aspects of the invention be in culture. In other aspects of the invention a host cells is prepared outside of an organism and is delivered into the organism. In some embodiments of the invention, a host cell is prepared at least in part in an organism. In some embodiments of the invention a host cell is prepared in an organism.

### Target Genes and Delivered Molecules

Target genes, also referred to herein as target nucleic acids, may include any nucleic acid sequence having an effect that is of interest to be modulated in a host cell and/or organism using methods of the invention. In some aspects of the invention, preselected agent is selected based on its effect on a target gene in a host cell. Thus, in some aspects of the invention a target gene in a host cell to be modified is identified and preselected agents are delivered into the host cell using methods of the invention and the delivered preselected agents modify the target gene. In some aspects of the invention a target gene comprises DNA, which may be double-stranded DNA or single-stranded DNA. A gene selected as target gene for editing using preselected agents delivered into a host cell may alter a nucleic acid sequence in the genome of a host cell.

Assays described herein, and others known in the art, can be used to determine whether a preselected agent is delivered and whether it is functions within the host cell in a manner in that results in a desired effect on the host cell. For example, though not intended to be limiting, if a desired effect on a target gene in a host cell is to inhibit or suppress a target gene's expression, assays can be performed to determine whether or not the one more delivered preselected agents are effective to reduce transcription or expression of the target gene. Means of assessing a cell for properties and function after delivery of one or more agents are known in the art and can be used in combination with methods and compositions of the invention to assess efficacy of delivery and function of molecules in host cells and organisms.

Preselected agents that can be delivered and utilized in host cells according to some aspects of the invention may include DNA binding proteins and functional variants thereof. In certain aspects of the invention, a DNA-binding protein may be a nucleic acid-guided DNA binding protein. Non-limiting examples of types of nucleic acid DNA-binding proteins that may be delivered in some embodiments of the invention include: RNA-guided DNA-binding proteins and DNA-guided DNA-binding proteins. DNA binding proteins are known in the art, and include, but are not limited to: naturally occurring DNA binding proteins, a non-limiting example of which is a Cas9 protein, which has nuclease activity and cuts double stranded DNA. Cas9 proteins and Type II CRISPR systems are well documented in the art. (See for example, Makarova et al., Nature Reviews, Microbiology, Vol. 9, June 2011, pp. 467-477, the content of which is incorporated by reference herein in its entirety.) As used herein, the term “DNA binding protein having nuclease activity” refers to DNA binding proteins having nuclease activity and also functional variants thereof. In a non-limiting example, in some embodiments of the invention, Cas9, and may be used in methods of the invention as an RNA-guided DNA binding protein having nuclease activity. Functional variants of Cas9 can also be used in methods and compositions of the invention. A functional variant of Cas9 differs in amino acid sequence from Cas9, referred to as the variant's “parent” sequence, while retaining from a least a portion to all of the nuclease activity of its parent protein.

In some embodiments, a preselected agent that is delivered into a host cell may comprise a DNA-guided DNA-binding nuclease. Information on identification and use of DNA-guided binding proteins, for example in DNA-guided genome editing systems, is available in the art (Gao, F., et al., Nature Biotech online publication, May 2, 2016: doi:10.1038/nbt.3547, the content of which is incorporated herein by reference in its entirety).

A DNA binding protein having nuclease activity function to cut double stranded DNA that may be used delivered in aspects of methods of the invention and may comprise a DNA binding protein that have one or more polypeptide sequences exhibiting nuclease activity. A DNA binding protein with multiple regions that have nuclease activity may comprise two separate nuclease domains, each of which functions to cut a particular strand of a double-stranded DNA. Polypeptide sequences that have nuclease activity are known in the art, and non-limiting examples include: a McrA-HNH nuclease related domain and a RuvC-like nuclease domain, or functional variants thereof. In S. pyogenes, a Cas9 DNA binding protein creates a blunt-ended double-stranded break that is mediated by two catalytic domains in the Cas9 binding protein: an HNH domain that cleaves the complementary strand of the DNA and a RuvC-like domain that cleaves the non-complementary strand. [See Jinke et al., Science 337, 816-821 (2012), the content of which is incorporated by reference herein in its entirety]. Cas9 proteins are known to exist in many Type II CRISPR systems, see for example, Makarova et al., Nature Reviews, Microbiology, Vol. 9, June 2011, pp. 467-477, supplemental information, the content of which is incorporated herein by reference in its entirety. The Cas9 protein may be referred by one of skill in the art in the literature as Csn1. Alternatives to Cas9 include but are not limited to Cpf1 proteins from Type V CRISPR systems (See for example Zetsche et al., Cpf1 Is a Single RNA-Guided Endonuclease of a Class 2 CRISPR-Cas System, Cell (2015), //dx.doi.org/10.1016/j.cell.2015.09.038). In certain aspects of the invention, a DNA binding protein that does not have nuclease activity may be delivered into a host cell.

Methods of the invention, in part, may also include delivery of one or more additional molecules into a host cell of the invention. A non-limiting example of such a molecule is a guide nucleic acid molecule, such as: guide RNAs and guide DNAs. Information relating to guide DNAs can be found in Gao, F., et al., Nature Biotech online publication, May 2, 2016: doi:10.1038/nbt.3547, the content of which is incorporated herein by reference in its entirety. Guide RNAs are also referred to herein as short guide RNAs, sgRNAs, and gRNAs, as well as crRNAs for certain nucleases such as Cpf1. A guide RNA is designed and selected such that it is complementary to a DNA sequence of the selected target gene in the genome of a cell, and so the guide RNA acts in complex with a DNA binding protein, or variant thereof to direct degradation of the complementary sequence within the target gene.

In some aspects of the invention methods are provided that can be used to prepare a host cell in which an exogenous nucleic acid sequence is delivered into the cell, and is expressed in the cell to produce a nucleic acid-guided DNA binding protein having nuclease activity, and one or more guide nucleic acids. In a non-limiting example: a vector comprising a sequence encoding the one or more guide RNAs and the RNA-guided DNA binding protein may be designed and delivered into a host cell. In certain aspects of the invention, expression of a vector sequence that is delivered into a host cell results in production of a complex of the RNA-guided DNA binding protein and guide RNAs that is directed by the guide RNA(s) to the preselected target gene, where the complex co-localizes to, or bind with, the target gene and the target gene is cleaved in a site-specific manner by the nuclease activity of the RNA guided DNA binding protein.

Methods of designing guide RNAs to direct an RNA-guided DNA binding protein to a selected target gene are known in the art. Guide RNAs can be designed, prepared, tested, and selected for delivery into a host cell of the invention and subsequent use to modify the genome of the host cell. Methods and compositions provided herein, can be used in conjunction with knowledge in the art relating to DNA binding, vector preparation and use, RNA-guided DNA binding proteins, CRISPR system components and implementation, etc. Methods of the invention also can be used to determine activity of delivered preselected agents, for example, though not intended to be limited, using a fluorescent reporter assay.

It will be understood that delivery of a preselected agent into a host cell may in some embodiments mean delivery of a nucleic acid that encodes the preselected agent wherein when in the host cell the preselected agent is expressed. In certain aspects of the invention, delivery of a preselected agent into a host cell means delivery of that agent in expressed form, for example as a polypeptide.

### Additional Components

Methods of the invention, in part, include design, construction, and use of additional sequences that may be included in a vector delivered to a host cell using methods described herein. Sequences such as: promoter sequences, enhancer sequences, 3′ untranslated region (3′UTR) sequences can be included, therapeutic molecules, toxins, detectable labels, etc. may be delivered using methods of the invention. Those skilled in the art will understand how to selected and prepare preselected agents based on methods, components, and strategies disclosed herein and art-known methods and components.

The terms “protein” and “polypeptide” are used interchangeably herein as are the terms “polynucleotide” and “nucleic acid” sequence. A nucleic acid sequence may comprise genetic material including, but not limited to: RNA, DNA, mRNA, cDNA, etc. As used herein with respect to polypeptides, proteins, or fragments thereof, and polynucleotides that encode such polypeptides the term “exogenous” means the one that has been introduced into a cell, cell line, organism, or organism strain and not naturally present in the wild-type background of the cell or organism strain.

In certain embodiments of the invention, a polypeptide or nucleic acid variant may be a polypeptide or nucleic acid, respectively that is modified from its “parent” polypeptide or nucleic acid sequence. Variant polypeptides and nucleic acids can be tested for one or more activities (e.g., delivery to a target gene, suppression of a target gene, etc.) to determine which variants are possess desired functionality for delivery and activity using methods of the invention. The skilled artisan will also realize that conservative amino acid substitutions may be made in a polypeptide, for example in a Cas9 polypeptide, to design and construct a functional variant useful for delivery into a host cell according to methods of the invention. As used herein the term “functional variant” used in relation to polypeptides is a variant that retains a functional capability of the parent polypeptide. As used herein, a “conservative amino acid substitution” refers to an amino acid substitution that does not alter the relative charge or size characteristics of the polypeptide in which the amino acid substitution is made. Conservative substitutions of amino acids may, in some embodiments of the invention, include substitutions made amongst amino acids within the following groups: (a) M, I, L, V; (b) F, Y, W; (c) K, R, H; (d) A, G; (e) S, T; (f) Q, N; and (g) E, D. Polypeptide variants can be prepared according to methods for altering polypeptide sequence and known to one of ordinary skill in the art such. Non-limiting examples of functional variants of polypeptides that can be delivered into cells using methods of the invention are functional variants of a Cas9 polypeptide, functional variants of detectable label sequences, etc.

As used herein the term “variant” in reference to a polynucleotide or polypeptide sequence refers to a change of 1, 2, 3, 4, 5, 6, 7, 8, 9, 10, or more nucleic acids or amino acids, respectively, in the sequence as compared to the corresponding parent sequence. For example, though not intended to be limiting, a variant guide RNA sequence may be identical to that of its parent guide RNA sequence except that 1, 2, 3, 4, 5, 6, 7, 8, 9, 10, or more nucleic acid substitutions, deletions, insertions, or combinations thereof, and thus is a variant of the parent guide RNA. In another non-limiting example, the amino acid sequence of a variant Cas9 nuclease polypeptide may be identical to that of its parent Cas9 nuclease except that it has 1, 2, 3, 4, 5, 6, 7, 8, 9, 10, or more amino acid substitutions, deletions, insertions, or combinations thereof, and thus is a variant of the parent Cas9 nuclease. Certain methods of the invention include delivering functional variants of components described herein, such as guide nucleic acids, guide RNAs, and guide DNAs. Methods provided herein, and other art-known methods can be used to prepare candidate guide sequences that can be tested for function and to determine whether they retain sufficient activity for use when delivered into and utilized within a host cell.

Methods of the invention provide means to test for activity and function of variant sequences and to determine whether a variant is a functional variant and is suitable for inclusion in a host cell of the invention. Suitability can, in some aspects of methods of the invention, be based on one or more characteristics such as: expression; cell localization; gene-cutting activity, efficacy in modulating activity of a target gene, etc. Functional variant polypeptides and functional variant polynucleotides that may be delivered and use in methods of the invention may be amino acid and nucleic acid sequences that have at least 75%, 80%, 85%, 90%, 95%, 96%, 97%, 98%, or 99% identity to their parent amino acid or nucleic acid sequence, respectively.

Art-known methods can be used to assess relative sequence identity between two amino acid or nucleic acid sequences. For example, two sequences may be aligned for optimal comparison purposes, and the amino acid residues or nucleic acids at corresponding positions can be compared. When a position in one sequence is occupied by the same amino acid residue, or nucleic acid as the corresponding position in the other sequence, then the molecules have identity/similarity at that position. The percent identity or percent similarity between the two sequences is a function of the number of identical positions shared by the sequences (i.e., % identity or % similarity=number of identical positions/total number of positions×100). Such an alignment can be performed using any one of a number of well-known computer algorithms designed and used in the art for such a purpose. It will be understood that a variant polypeptide or polynucleotide sequence may be shorter or longer than their parent polypeptide and polynucleotide sequence, respectively. The term “identity” as used herein in reference to comparisons between sequences may also be referred to as “homology”.

### Delivery

Components may be delivered into a cell using the teaching provided herein in combination with routine molecular biology techniques. In certain aspects of the invention, vectors are used to deliver one or more preselected agents into a host cell. As used herein, the term “vector” used in reference to delivery of components refers to a polynucleotide molecule capable of transporting between different genetic environments another nucleic acid to which it has been operatively linked. One type of vector is an episome, i.e., a nucleic acid molecule capable of extra-chromosomal replication. Some useful vectors are those capable of autonomous replication and/or expression of nucleic acids to which they are linked. Vectors capable of directing the expression of genes to which they are operatively linked may be referred to herein as “expression vectors”. Other useful vectors, include, but are not limited to viruses such as lentiviruses, retroviruses, adenoviruses, and phages. Vectors useful in some methods of the invention can genetically insert one or more of a gene drive cassette into a dividing or a non-dividing cell and can enable delivery of one or more preselected agents into an in vivo or in vitro host cell.

### Hosts, Cells, Cell Lines, and Organisms

One or more methods of the invention for delivering preselected agents and modifying a host cell as described here can be applied to preselected agents and modifications into a host cell or organism. A host cell or organism is one to which exogenous biomolecule is expressed and/or to which one or more preselected agents are delivered. In some aspects of the invention, a host cell and its progeny are understood to be member of a cell strain that includes modifications resulted from the delivered preselected agent(s). Similarly, a host organism and its progeny that include genome edits resulting from preselected agents delivered into the host cell or organism may be referred to as an organism of a host strain, or host strain organisms, or simply as a host strain. A mutant lineage of an organism that is prepared using delivered preselected agents according to the invention may also be referred to as a “strain”.

Non-limiting examples of stages of cells to which one or more preselected agents may be delivered or included are: embryonic cells, germline cells, gametes, cells that can give rise to a gamete, somatic cells, zygotes, pre-meiotic cells, post-meiotic cells, fully-differentiated cells, and mature cells. Cells at one or more of these stages may be isolated cells, cells in cell lines, cells in cell, tissue, or organ culture, cells that are within an organism. In certain embodiments of the invention, a cell is a zygote, a gamete, a cell that is able to give rise to a gamete, a germline cell, etc.

Methods of the invention with which to deliver preselected agents into a host cell and/or organism may be used in host cells from various organisms. In some aspects of the invention, a cell or organism is a vertebrate or an invertebrate cell or organism. In some aspects of the invention, a cell or organism is a mammal and in certain embodiments is a human, non-human primate, cow, house, rat, mouse, etc. In certain aspects of the invention, a cell or organism is a eukaryotic or prokaryotic cell or organism. Non-limiting examples of organisms to which delivery of preselected agents into cells according to methods of the invention may be used are: insects, fish, reptiles, amphibians, mammals, birds, protozoa, annelids, mollusks, echinoderms, flatworms, coelenterates, and arthropods, including arachnids, crustaceans, insects, and myriapods. In some aspects of the invention a yolk bearing organism and in certain aspects an organism is a non-yolk bearing organism.

Some embodiments of methods of the invention include delivering one or more preselected agents into a plant or plant cell, including: monocots and dicots, weeds, invasive plants, poisonous plants, aquatic plants, terrestrial plants, recombinant plants, etc.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Example 1

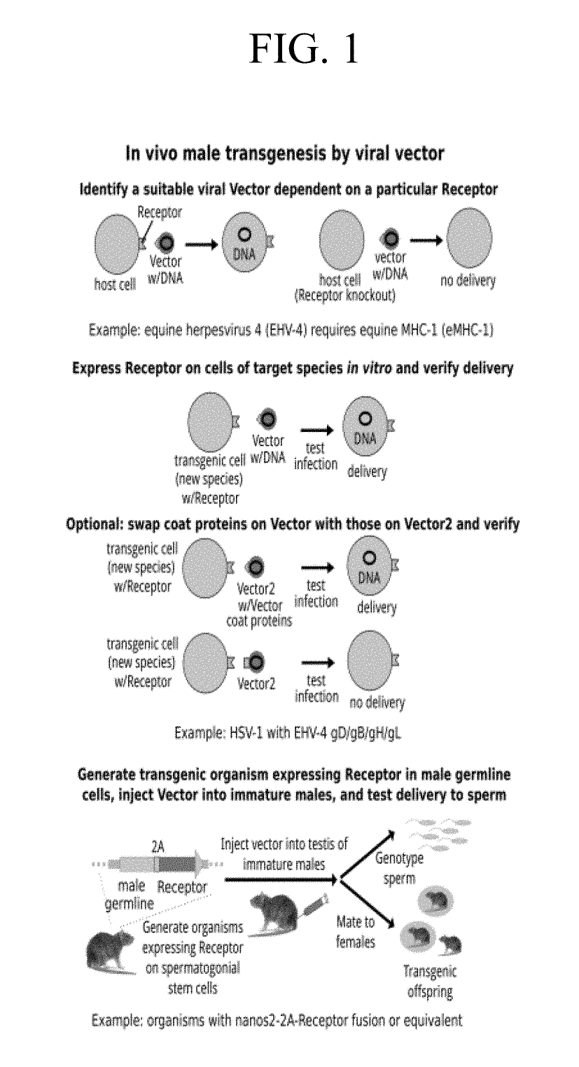

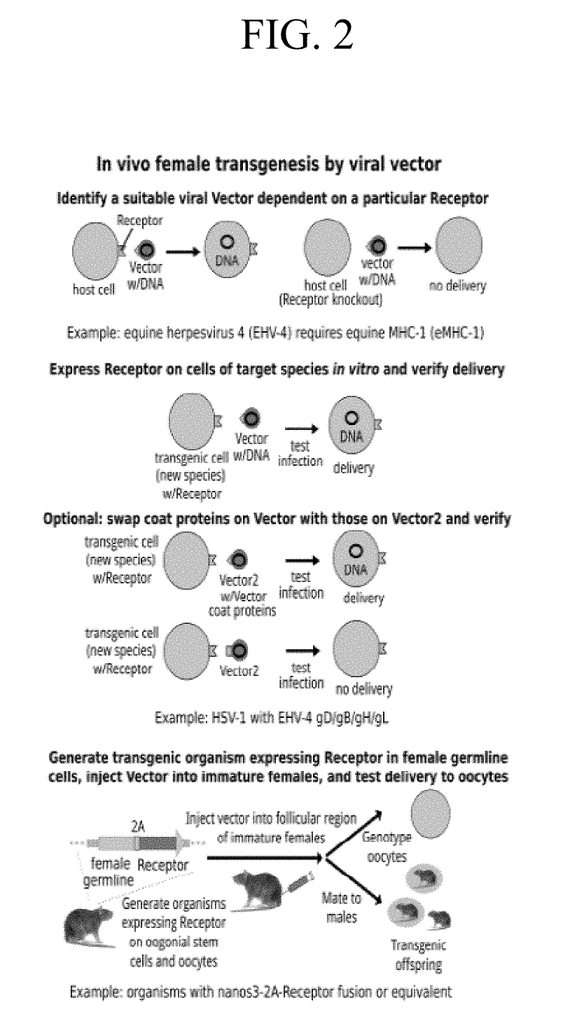

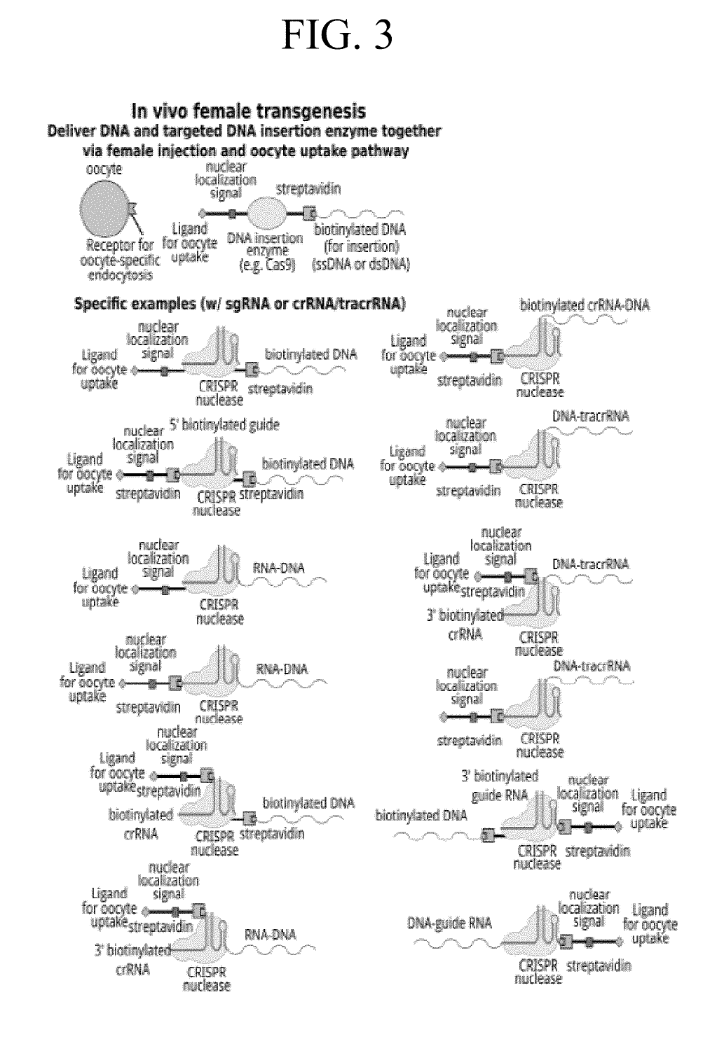

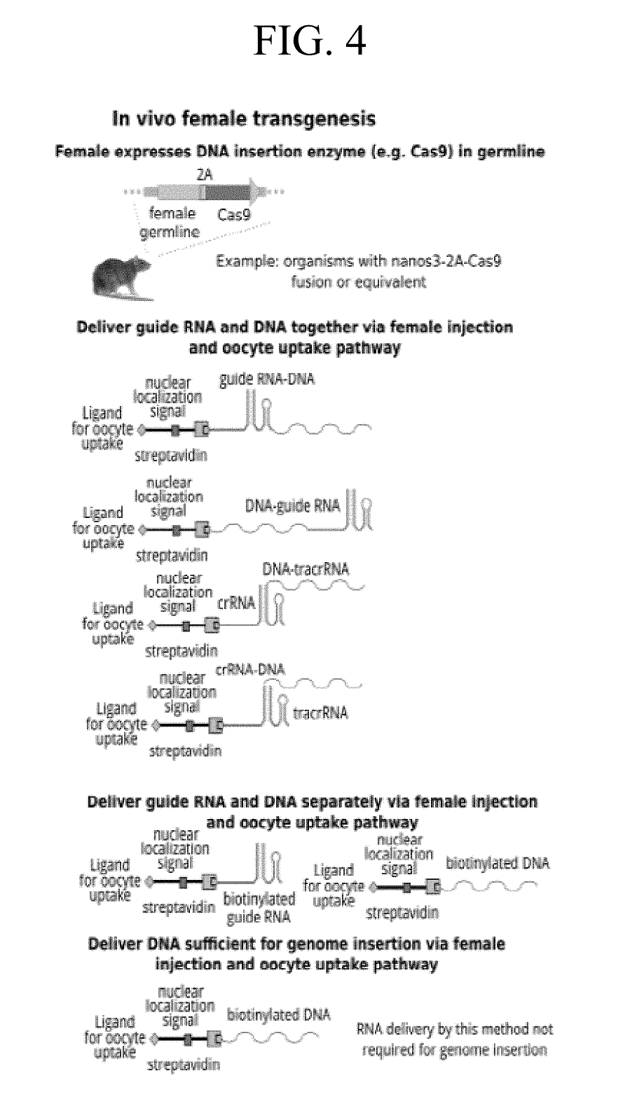

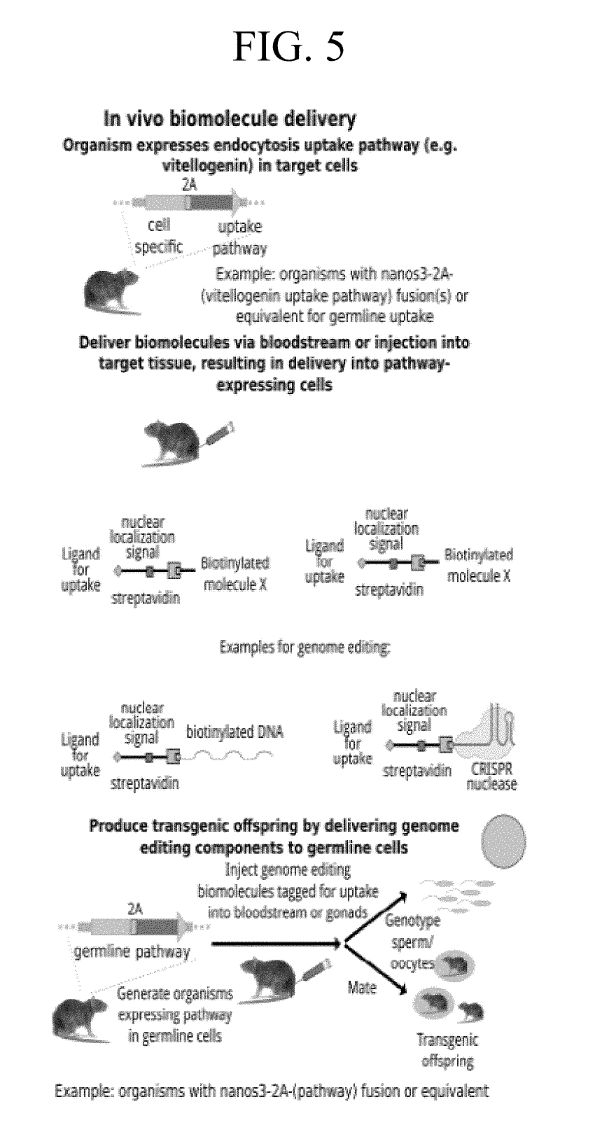

Delivery is a limiting bottleneck for most research involving multicellular organisms, including for generating transgenic organisms. Experiments are now performed that include novel methods of accomplishing in vivo biomolecule delivery, specifically focused on but not limited to editing the germline of adult male and female organisms. Schematic diagrams detailing embodiments of methods of the invention are provided as FIGS. 1-5. FIGS. 1-5 provide non-limiting examples of how methods of the invention can be used to (1) prepare and utilize host cells; (2) alter the genome of a host cell; (3) generate progeny that include modifications of the host cell; and (4) cross progeny that include modifications of the host cell.

### Method 1: Viral Vectors

Viruses specialize in delivering nucleic acids into cells, but the germlines of multicellular organisms are difficult to infect. Some research groups have reportedly transduced spermatogonial cells of the male germline by injecting lentiviral vectors into the seminiferous tubules of immature males, whose germline defenses against viruses have not yet fully formed [1]. Because lentiviruses do not transduce the germline particularly well, this method has a low success rate, albeit one that can be boosted by pseudotyping the lentivector with VSV-G [2, 3], a coat protein that allows transduction of all cell types. However, this is presumably inefficient, as most cells in the testis are not germline and consequently will soak up most of the lentivector. Moreover, lentiviral vectors can only carry about 8 kilobases of DNA or less, which is not enough for most applications.

Methods have been developed that render herpesviral vectors, which can carry over 100 kilobases of DNA, able to efficiently and exclusively transduce transgenic cells.

(1) The method involves first identifying a herpes Virus from another species which cannot normally transduce any cells of a target organism.
(2) Second, identify the target Receptor this Virus uses to enter the cell in its normal host organism (e.g. equine major histocompatibility complex 1 [eMHC-1] protein for equine herpesvirus 4 [EHV-4] [4]).
(3) Third, engineer cells of the target organism to express the Receptor in the cell type of interest. For transgenesis, this requires making a transgenic organism that expresses the Receptor in the germline (for example, expressing eMHC-1 under the control of the DazL promoter).
(4) Fourth, verify that the Virus can transduce the engineered target cells expressing the Receptor, but not wild-type cells.
(5) Fifth, insert the desired DNA to be delivered into the genome of the Virus (e.g. create a viral vector), typically including the CRISPR system or other enzyme capable of catalyzing genome insertion, and purify the virus to obtain a higher tier. Optionally, this may include exchanging the envelope proteins of a related viral vector, such as HSV-1, with the entry proteins of the Virus to specifically target the Receptor rather than the original tropism of the vector.
(6) The viral vector is injected at high tier into the target tissues of organisms (gonadal tissue in the case of transgenesis), where they transduce the transgene-expressing cells efficiently using the transgenic Receptor. Details of the procedures are shown in FIGS. 1-5 herein.

### Method 2: Oocyte Uptake

In many species, the pathways governing uptake of proteins and nutrients by the developing oocyte have been mapped. In vitellogenic species (those that make a yolk), it is possible to deliver biomolecules into the oocyte by fusing the amino acid signaling tag of vitellogen to the target molecule [5]. Injecting females with tagged DNA and tagged DNA editing enzymes into the oocytes in this manner could produce transgenic organisms. Limitations of these methods reduce their efficacy.

Methods are used in that differ from prior methods and greatly improve uptake of molecules into oocytes.

(1) First, instead of inefficiently delivering the DNA editing enzyme (e.g. a CRISPR nuclease) via the bloodstream and uptake pathway, a transgenic organism is generated that expresses the DNA editing enzyme in the oocyte. A DNA tagged with the amino acid sequence corresponding to oocyte uptake (e.g. vitellogenin) is then delivered via the female bloodstream, optionally with tagged RNA or other biomolecules that will direct editing to particular site in the genome. The DNA editing enzyme will then act to insert the target sequence.
(2) Second, to increase the efficiency of chromosomal insertion, the DNA editing enzyme and the DNA to be inserted are delivered as a single molecule fused to one or more copies of the oocyte uptake tag. Co-localization of DNA and editing machinery ensures that the DNA is proximal to the enzyme upon editing, thereby increasing the likelihood that it will be inserted correctly. Numerous methods of attaching DNA to enzymes are known to those in the art, and can be used in methods of the invention.
(3) Third, to increase the efficiency of information transfer into the cell by generating a cisgenic or transgenic organism (e.g. a rodent) that expresses an endocytosis pathway for biomolecule uptake native to the same species or to a different species (e.g. the vitellogenin pathway common to egg-laying species that require yolk protein delivery to oocytes that is not found in mammals).

Each of the delivery methods of the invention as described herein increase efficacy of delivery of molecules into cells and organisms. Genome editing is performed in generated cells and organisms as described herein. Oocytes into which molecules have been delivered can be developed into mature organisms. The mature organisms are crossed with wild-type, transgenic, and/or other altered organisms to produce progeny that include genome edits set forth in the oocyte and/or other cells altered using methods and compounds of the invention.

### REFERENCES FOR EXAMPLE 1

1. Kanatsu-Shinohara, M., et al., “Transgenic Mice Produced by Retroviral Transduction of Male Germ Line Stem Cells in Vivo” Biology of reproduction 71, no. 4 (2004): 1202-1207.
2. Sehgal, L., et al. “Lentiviral Mediated Transgenesis by in Vivo Manipulation of Spermatogonial Stem Cells” PloS one 6, no. 7 (2011): e21975.
3. Qin, J., et al. “An Efficient Strategy for Generation of Transgenic Mice by Lentiviral Transduction of Male Germline Stem Cells in Vivo” Journal of animal science and biotechnology 6, (2015): 59.
4. Azab, W. and Osterrieder, N. “Glycoproteins D of Equine Herpesvirus Type 1 (EHV-1) and EHV-4 Determine Cellular Tropism Independently of Integrins” Journal of virology (2012): February; 86(4); 2031-44.
5. Rungger, D., et al., “Oocyte Shuttle, a Recombinant Protein Transporting Donor DNA into the Xenopus Oocyte in Situ” Biology open 6, no. 2 (2017): 290-295.

### EQUIVALENTS

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated herein in their entirety herein by reference.

What is claimed is:

1. A composition comprising a host cell, wherein the host cell comprises:
a) a preselected biomolecule expressed on the surface of the host cell, wherein the expressed preselected exogenous biomolecule is capable of enabling delivery of one or more preselected agents into the host cell; and
b) one or more preselected agents comprising at least one of a: nucleic acid molecule, DNA molecule, RNA molecule, nucleic acid-editing molecule, and genome-editing molecule.

2-3. (canceled)

4. The composition of claim 1, wherein the preselected biomolecule comprises a receptor molecule, optionally, wherein the receptor molecule comprises one or more of a low-density lipoprotein (LDL) receptor, a very low-density lipoprotein receptor a membrane channel, and a membrane pore and optionally where the LDL receptor is a vitellogenin receptor.

5-43. (canceled)

44. A method of delivering one or more exogenous agents into an oocyte of an original species, the method comprising:
a) preparing a transgenic oocyte comprising an expressed exogenous low-density lipoprotein (LDL) receptor in a host oocyte of an original species, wherein (i) the exogenous LDL receptor is not naturally expressed in an oocyte of the original species and (ii) the expressed exogenous LDL receptor is displayed on the surface of the transgenic oocyte;
b) contacting the expressed exogenous LDL receptor with a ligand complex comprising:
(iii) an LDL receptor ligand molecule that specifically interacts with the expressed exogenous LDL receptor and (iv) one or more preselected agents in association with the LDL receptor ligand molecule, wherein the contacted expressed exogenous LDL receptor is capable of enabling delivery of the one or more preselected agents into the transgenic oocyte; and
c) delivering the one or more preselected agents into the transgenic oocyte displaying the expressed exogenous LDL receptor.

45. The method of claim 44, wherein the LDL receptor ligand molecule is in association with at least one first preselected agent that is in association with a second preselected agent, wherein the specific interaction of the LDL receptor ligand molecule with the expressed exogenous LDL receptor delivers the second preselected agent into the transgenic oocyte.

46. (canceled)

47. The method of claim 44, wherein the expressed exogenous LDL receptor comprises an exogenous vitellogenin receptor and the LDL receptor ligand molecule comprises a vitellogenin receptor ligand that specifically interacts with the expressed exogenous vitellogenin receptor.

48. The method of claim 47, where the vitellogenin receptor ligand is attached to at least one preselected agent and the interaction between the expressed exogenous vitellogenin receptor molecule and the vitellogenin receptor ligand delivers at least one of the preselected agents into the transgenic oocyte.

49. The method of claim 47, wherein the vitellogenin receptor ligand is attached to at least one first preselected agent that is in association with a second preselected agent and the interaction of the vitellogenin receptor ligand with the expressed preselected exogenous vitellogenin receptor delivers the second preselected agent into the transgenic oocyte.

50-53. (canceled)

54. The method of claim 44, further comprising delivering at least one nucleic acid and nucleic acid-editing molecule into the transgenic oocyte, optionally wherein the nucleic acid-editing molecule is a genome-editing molecule, optionally wherein a means of delivering the nucleic acid-editing molecule comprises delivering into the transgenic oocyte a nucleic acid molecule encoding the nucleic acid-editing molecule, and expressing the nucleic acid-editing molecule in the transgenic oocyte.

55. (canceled)

56. The method of claim 54, wherein the nucleic acid encoding the nucleic acid-editing molecule further comprises a nucleic acid encoding a molecule to be inserted into the genome of the transgenic oocyte.

57-58. (canceled)

59. The method of claim 44, further comprising editing the genome of the transgenic oocyte.

60. The method of claim 44, further comprising developing an organism from the transgenic oocyte.

61-67. (canceled)

68. A method of delivering one or more exogenous agents into an oocyte of an original species, the method comprising:
a) contacting a host oocyte displaying an expressed endogenous low-density lipoprotein (LDL) receptor on its surface, with a ligand complex comprising: (i) an LDL receptor ligand molecule that specifically interacts with the expressed endogenous LDL receptor and (ii) one or more preselected agents in association with the LDL receptor ligand molecule, wherein the contacted expressed endogenous LDL receptor is capable of enabling delivery of the one or more preselected agents into the host oocyte; and
b) delivering the one or more preselected agents into the host oocyte displaying the expressed exogenous LDL receptor on its surface.

69. The method of claim 68, wherein the LDL receptor ligand molecule is in association with at least one first preselected agent that is in association with a second preselected agent, wherein the specific interaction of the LDL receptor ligand molecule with the expressed endogenous LDL receptor delivers the second preselected agent into the host oocyte.

70. (canceled)

71. The method of claim 68, wherein the expressed endogenous LDL receptor comprises an endogenous vitellogenin receptor and the LDL receptor ligand molecule comprises an endogenous vitellogenin receptor ligand that specifically interacts with the expressed endogenous vitellogenin receptor.

72. The method of claim 71, where the vitellogenin receptor ligand is attached to at least one preselected agent and the interaction between the expressed endogenous vitellogenin receptor molecule and the vitellogenin receptor ligand delivers at least one of the preselected agents into the host oocyte.

73. The method of claim 71, wherein the vitellogenin receptor ligand is attached to at least one first preselected agent that is in association with a second preselected agent and the interaction of the vitellogenin receptor ligand with the expressed endogenous vitellogenin receptor delivers the second preselected agent into the host oocyte.

74-77. (canceled)

78. The method of claim 68, further comprising delivering at least one nucleic acid and at least one nucleic acid-editing molecule into the host oocyte, optionally wherein the nucleic acid-editing molecule is a genome-editing molecule, optionally wherein a means of delivering the nucleic acid-editing molecule comprises delivering into the host oocyte a nucleic acid molecule encoding the nucleic acid-editing molecule, and expressing the nucleic acid-editing molecule in the host oocyte.

79. (canceled)

80. The method of claim 78, wherein the nucleic acid encoding the nucleic acid-editing molecule further comprises a nucleic acid encoding a molecule to be inserted into the genome of the host oocyte.

81-82. (canceled)

83. The method of claim 68, further comprising editing the genome of the contacted host oocyte.

84. The method of claim 68, further comprising developing an organism from the contacted host oocyte.

85-139. (canceled)
